(19) United States
(12) Patent Application Publication
(10) Pub. No.: US 2014/0147381 A1
(43) Pub. Date: May 29, 2014

(54) 89ZR COMPOUNDS, TO INCLUDE SOMATOSTATIN, APPARATUS AND PRODUCTS COMPRISING SUCH COMPOUNDS, METHODS OF MAKING SAME, AND METHODS OF USING SAME FOR RADIO IMAGING AND/OR TREATMENT

(71) Applicant: GREGORY DAVID ESPENAN, Metairie, LA (US)

(72) Inventor: GREGORY DAVID ESPENAN, Metairie, LA (US)

(21) Appl. No.: 13/689,558

(22) Filed: Nov. 29, 2012

(51) Int. Cl. A61K 51/08; C07K 1/13

(52) U.S. Cl. CPC A61K 51/083; C07K 1/13
USPC 424/1.45; 530/311

(57) ABSTRACT

⁸⁹Zr radiolabeled somatostatin receptors and other receptors, methods of their making, and method of their using for imaging and treatment of somatostatin receptor bearing tumors and other receptor bearing tumors.

### RELATED APPLICATION DATA

Not applicable.

### BACKGROUND OF THE INVENTION

1. Field of the Invention

The present invention relates to radioisotope compositions, apparatus and products comprising such compositions, and methods relating to such compositions, apparatus and products. In another aspect, the present invention relates to radioisotope compositions, apparatus and products comprising such compositions, and methods of making or using such compositions, apparatus and products. In even another aspect, the present invention relates to radioisotope compositions with affinity for certain receptors, apparatus and products comprising such compositions, and methods of making or using such compositions, apparatus and products. In still another aspect, the present invention relates to radioisotope compositions with affinity for certain receptors, apparatus and products comprising such compositions, methods of making such compositions, apparatus and products, imaging methods using such compositions, apparatus and products for imaging, and treatment methods using such compositions, apparatus. In yet another aspect, the present invention relates to ⁸⁹Zr radioisotope compositions comprising somatostatins, apparatus and products comprising such compositions, methods of making or using such compositions, apparatus and products.

2. Description of the Related Art

The presence of somatostatin receptors has been demonstrated in a variety of human tumors, e.g. pituitary tumors, central nervous system tumors, breast tumors, gastro-enteropancreatic tumors and their metastases. Some of them are small or slow-growing tumors which are difficult to precisely localize by conventional diagnosis methods.

Currently, patients who have somatostatin receptor bearing tumors are imaged with a radiolabelled drug called Octreoscan-111In-DTPA-Penetetreotide. Most commonly, the patient is injected with 6 mCi and sequentially imaged over several days utilizing a gamma camera.

With technological advancements over the past number of years, Positron Emission Tomography (PET) units have become more prevalent in medical treatment. In fact, it is safe to say that PET has become one of the most prominent functional imaging modalities in diagnostic medicine, with very high sensitivity (fmoles), high resolution (4-10 mm) and tissue accretion that can be adequately quantitated.

Among other advantages, PET allows for imaging of smaller lesions and when combined and fused with computed tomography (CT) provides superior localization. Further, PET has a much higher signal to noise ratio than traditional imaging apparatus and methods. Perhaps the most commonly used radiotracer is 18FDG (Flourine-18 Deoxy-Glucose). However, FDG does not image somatostatin receptor bearing tumors well as often those tumors are slow growing and the FDG localizes in cells with high mitotic activity. Some experimentation has been performed with 68Ga labeled DOTA-Octreotate, however, the short half-life and prohibitive cost of this radioisotope has limited the use of this PET pharmaceutical. Moreover, these somatostatin receptor bearing tumors are currently imaged best when the non-tumor bound activity has had time to be removed biologically.

The following patents, applications, and/or publications are merely a few of the many relating to tumor imaging and/or treatment.

U.S. Pat. No. 5,753,627, issued May 19, 1998 to Albert, et al., discloses the use of certain complexedsomatostatin peptides for the invivo imaging of somatostatin receptor-positive tumors and metastasis. These Somatostatin peptides bear at least one chelating group for a detectable element, said chelating group being linked to an amino group of said peptide, and said amino group having no significant binding affinity for somatostatin receptors, in free or salt form, and are complexed with a detectable element and are useful as a pharmaceutical, e.g. a radiopharmaceutical for in vivo imaging of somatostatin receptor positive tumors or for therapy.

Arnold R, Simon B, Wied M. Treatment of neuroendocrine GEP tumours with somatostatin analogues: a review. Digestion 2000; 62 (Suppl 1):84-91, discloses that somatostatin and its long-acting analogues are effective in symptom control in patients with functionally active neuroendocrine GEP tumours, with several in vitro and in vivo reports suggesting that they are also able to control tumour growth. Simon et al. provides a critical review of published data on the effect of long-acting somatostatin analogues on symptom and growth control in patients with metastatic neuroendocrine GEP tumours. Simon reports results that with the exception of insulinoma and gastrinoma, octreotide acetate and other long-acting somatostatin formulations are currently the therapeutic principle of first choice to control hormone-mediated symptoms. Simon further reports that the consequences of gastric acid hypersecretion in patients with Zollinger-Ellison syndrome are best controlled by proton pump inhibitors. Simon further reports that available data on growth control indicate that stabilization of tumour growth seems to be the most beneficial antiproliferative effect occurring in up to 50% of patients, however that the effect is limited, and it is unknown which tumour entity responds best to longactingsomatostatin analogues. Simon proposes that additional studies in patients with known spontaneous tumour growth and avoiding a mix-up of different entities of neuroendocrine malignancies are necessary to identify subpopulations of neuroendocrine tumours which respond to long-acting somatostatin analogues in terms of longer lasting growth inhibition.

U.S. Pat. No. 6,180,082, issued Jan. 30, 2001, and U.S. Pat. No. 6,630,123, issued Oct. 7, 2003 both to Woltering et al, disclose methods to enhance tissue accumulation of radiolabeled compounds. The administration of a radioisotopic compound by infusion over a period of time greater than two hours, preferably greater than twelve hours, greatly increases the maximum radioactivity that accumulates in the target cell. The efficacy of the administration of the radiolabeled compound can be increased about five times higher than prior bolus injection or short infusion methods. This method enhances the tumor to background ratio by increasing the actual radioligand accumulated inside the target cells. This technique works for any radiolabeled compound whose cellular uptake is limited by a cellular process of either binding to a cellular receptor or to a transport protein. Once the radiolabeled compound is bound and internalized, the ability of an unlabeled compound to compete with the radioligand is markedly decreased. The primary factor governing residence time after internalization is the physical half-life of the radioisotope, not biologic half-life.

U.S. Pat. No. 6,465,613, issued Oct. 15, 2002, to Coy, et al., discloses novel hydrophilic somatostatin analogs that may be readily labeled with toxic or non-toxic detectable labels. These unlabeled and labeled analogs are useful for specifically targeting somatostatin receptor bearing cells, in particular neoplastic cells. Labeled analogs are useful, for example, for tumor localization and detection. Where labeled with a toxic label (e.g., radioactivity), the analogs are useful for the targeted delivery of toxicity to somatostatin receptor-bearing cells, in particular neoplastic cells. Also disclosed are methods for treating and detecting neoplasms, and methods for imaging somatostatin receptor-bearing cells. Taal B G, Visser O. Epidemiology of neuroendocrine tumours. Neuroendocrinology 2004; 80 (Suppl 1):3-7, discloses that neuroendocrine tumours account for only 0.5% of all malignancies, with the incidence being approximately 2/100,000 with a female preponderance under the age of 50 years due to appendiceal location, with the main primary sites are the gastrointestinal tract (62-67%) and the lung (22-27%). Taal et al. further discloses that presentation with metastatic disease accounts for 12-22%, and that in the last decades, the incidence has been rising, and that this rise might be due to more awareness, improved diagnostic tools or a change in definition. Taal et al. further report that most neuroendocrine tumours are mainly sporadic, but association with the multiple endocrine neoplasia type 1 syndrome and clustering within families is known, and that an increased risk of secondary cancers has been reported, but numbers are small. Taal et al. note that the 5-year survival is mainly associated with stage: 93% in local disease, 74% in regional disease and 19% in metastatic disease. In metastatic disease, survival increased since 1992, when treatment with octreotide became largely available in the Netherlands.

Raut C P, Kulke M H, Glickman J N, et al. Carcinoid tumors. CurrProbl Surg. 2006 Jun;43(6):383-450, disclose that octreotide administered subcutaneously every 8 hours is effective incontrolling the symptoms of carcinoid syndrome, and that longer acting somatostatin analogues lanreotide (administered every 10 to 14 days) and depot octreotide (administered monthly) are available. Raut et al. further disclose that biochemical response rates of 27% to 72% have been observed, but tumor responserates only range from 0% to 9%, that Interferon has been evaluated in several trials, with pooled data showing biochemical and tumor response rates of 40% and 12%, respectively. Raut et al note that trials with cytotoxic chemotherapy, as single agents or in combination, have been reported, and that the response rates, measured by either tumor regression or decrease in urinary 5-HIAA, were 0% to 29%. Raut et al also note that currently available biologic therapies and chemotherapy regimens have minimal efficacy in reducing tumor size, and that more specific targeted therapies employing agents such as thalidomide, bevacizumab, and sunitinib are under investigation.

U.S. Pat. No. 7,202,330, issued Apr. 10, 2007, to DeJong et al, discloses a RGD (Arg-Gly-Asp) coupled to (neuro)peptides. The invention relates to compounds having a binding affinity for both the Δvβ3 receptor and a (neuro)peptide receptor, in particular the somatostatin receptor, which compound comprises a first peptide part comprising at least once the amino acid sequence Arg-Gly-Asp, and a second peptide part coupled thereto, optionally via a linker, which second peptide part is a (neuro)peptide.

U.S. Patent Publication No. 2008/0193380, published Nov. 21, 2007, and U.S. Pat. No. 7,344,700, issued Mar. 18, 2008, both to Dalton et al, disclose a radiolabeled selective androgen receptor modulators and their use in prostate cancer imaging and therapy. The provided is a class of radiolabeled androgen receptor targeting agents (ARTA), useful for prostate cancer imaging and in treating or preventing prostate cancer. The agents define a new subclass of radiolabeled compounds, which are selective androgen receptor modulators (SARM), which demonstrate antiandrogenic activity of a nonsteroidal ligand for the androgen receptor, and/or which bind irreversibly to the androgen receptor. The present invention further provides methods for a) imaging of cancer in a subject, b) imaging an androgen receptor-containing tissue in a subject, c) in-vivo imaging in a subject, d) treating a subject suffering from prostate cancer, e) delaying the progression of prostate cancer in a subject suffering from prostate cancer, f) preventing the recurrence of prostate cancer in a subject suffering from prostate cancer, and g) treating the recurrence of prostate cancer in a subject suffering from prostate cancer, which comprise using the radio labeled compounds of the present invention. The present invention further provides a method of producing the radiolabeled SARM compounds, and precursor compounds useful in the preparation of the radiolabeled SARM compounds.

Yao J C, Hassan M, Phan A, et al. One hundred years after “carcinoid”: epidemiology of and prognostic factors for neuroendocrine tumors in 35,825 cases in the United States. J ClinOncol. 2008 Jun 20;26(18):3063-72, discloses that neuroendocrine tumors (NETs) are considered rare tumors and can produce a variety of hormones, and provides an examination of the epidemiology of and prognostic factors for NETs, because a thorough examination of neither had previously been performed. Specifically, the Surveillance, Epidemiology, and End Results (SEER) Program registries were searched to identify NET cases from 1973 to 2004, with the associated population data used for incidence and prevalence analyses. Yao et al. identified 35,618 patients with NETs, and observed a significant increase in the reported annual age-adjusted incidence of NETs from 1973 (1.09/100,000) to 2004 (5.25/100,000). Using the SEER 9 registry data, they estimated the 29-year limited-duration prevalence of NETs on January 1, 2004, to be 9,263, and estimated the 29-year limited-duration prevalence in the United States on that date was 103,312 cases (35/100,000). Yao et al. notes the most common primary tumor site varied by race, with the lung being the most common in white patients, and the rectum being the most common in Asian/Pacific Islander, American Indian/Alaskan Native, and African American patients. Additionally, survival duration varied by histologic grade. In multivariate analysis of patients with well-differentiated to moderately differentiated NETs, disease stage, primary tumor site, histologic grade, sex, race, age, and year of diagnosis were predictors of outcome (P<0.001). Yao et al. observed increased reported incidence of NETs and increased survival durations over time, suggesting that NETs are more prevalent than previously reported, and notes that clinicians need to be become familiar with the natural history and patterns of disease progression, which are characteristic of these tumors.

Jason P. Holland, D. Phil, YiauchungSheh, and Jason S. Lewis, Ph.D* Standardized methods for the production of high specific-activity zirconium-89, Nucl Med Biol. 2009 October; 36(7): 729-739, notes that zirconium-89 is an attractive metallo-radionuclide for use in immunoPET due to the favorable decay characteristics. Holland et al. report standardized methods for the routine production and isolation of high purity and high specific-activity 89Zr using a small cyclotron, with optimized cyclotron conditions providing high average yields of 1.52±0.11 mCi/μA·h at a proton beam energy of 15 MeV and current of 15 μA using a solid, commercially available 89Y-foil target (0.1 mm, 100% natural abundance). Specifically, 89Zr was isolated in high radionuclidic and radiochemical purity (>99.99%) as [⁸⁹Zr]-oxalate by using a solid-phase hydroxamate resin with >99.5% recovery of the radioactivity, and the effective specificactivity of ⁸⁹Zr was found to be in the range 5.28-13.43 mCi/μg (470-1195 Ci/mmol) of zirconium. Holland et al. also report wew methods for the facile production of [⁸⁹Zr]-chloride, with radiolabeling studies using the trihydroxamate ligand desferrioxamine B (DFO) providing 100% radiochemical yields in <15 min. at room temperature and in vitro stability measurements confirmed that [⁸⁹Zr]-DFO is stable with respect to ligand dissociation in human serum for >7 days. They further report that small-animal PET imaging studies have demonstrated that free ⁸⁹Zr(IV) ions administered as [⁸⁹Zr]-chloride accumulate in the liver whilst [⁸⁹Zr]-DFO is excreted rapidly via the kidneys within <20 min, and that these results have important implication for the analysis of immunoPET imaging of ⁸⁹Zr-labeled monoclonal antibodies. They state that the detailed methods described can be easily translated to other radiochemistry facilities and will facilitate the use of ⁸⁹Zr in both basic science and clinical investigations.

Prasad V, Baum RP. Biodistribution of the Ga-68 labeled somatostatin analogue DOTA-NOC in patients with neuroendocrine tumors: characterization of uptake in normal organs and tumor lesions; Q J Nucl Med Mol Imaging. 2010 Feb; 54(1):61-7, discloses that ⁶⁸Ga DOTA-NOC is an excellent tracer for imaging somotostatin receptor positive tumors, which, due to the high target to non-target rations, allows that detection of very small lesions, especially of the lymph node and bone metastases.

U.S. Pat. No. 7,662,783, issued Feb. 16, 2010, to Brooks et al, discloses a CLK-peptide and SLK-peptide. The invention describes methods for inhibiting angiogenesis in a tissue by administering an antagonist that specifically binds to a proteolyzed or denatured collagen type-IV with substantially greater affinity than to the native triple helical form of collagen type-IV. Methods utilizing such antagonists for therapeutic treatment of tumor growth, tumor metastasis or of restenosis also are described, as are methods to use such antagonists as diagnostic markers of angiogenesis in normal or diseased tissues both in vivo and ex vivo. The invention further describes methods for treating tumors using said antagonists in combination with radiation therapy and therapies comprising the antagonists and radiation treatment.

U.S. Pat. No. 8,053,415, issued Nov. 8, 2011, to Samuel et al., discloses compounds having RD targeting motifs. The present invention provides compounds that have motifs that target the compounds to cells that express integrins. In particular, the compounds have peptides with one or more RD motifs conjugated to an agent selected from an imaging agent and a targeting agent. The compounds may be used to detect, monitor and treat a variety of disorders mediated by integrins.

U.S. Pat. No. 8,153,100, issued Apr. 10, 2012, to McBride et al, discloses methods and compositions for F-18 labeling of proteins, peptides and other molecules. The present application discloses compositions and methods of synthesis and use of 18F or 19F labeled molecules of use in PET or MRI imaging. The labeled molecules may be peptides or proteins, although other types of molecules may be labeled. Preferably, the 18F or 19F is conjugated to a targeting molecule by formation of a metal complex and binding of the 18F- or 19F-metal complex to a chelating moiety. Alternatively, the metal may first be conjugated to the chelating group and subsequently the 18F or 19F bound to the metal. In other embodiments, the 18F or 19F labeled moiety may comprise a targetable construct used in combination with a bispecific antibody to target a disease-associated antigen. The 18F or 19F labeled targetable construct peptides are stable in serum at 37° C. for a sufficient time to perform PET or MRI imaging.

All of the patents, applications and publications cited in this specification, are herein incorporated by reference.

In spite of the prior art, there is a need in the art for methods, apparatus and products relating to the imaging and/or treatment of tumors.

There is another need in the art for methods, apparatus and products relating to imaging and/or treatment of somatostatin receptor or other receptor bearing tumors.

There is even another need in the art for methods, apparatus and products relating to the radio imaging of and/or treatment of somatostatin receptor or other receptor bearing tumors.

These and other needs in the art will become apparent to those of skill in the art upon review of this specification, including its drawings and claims.

### SUMMARY OF THE INVENTION

It is an object of the present invention to provide for methods, apparatus and products relating to the imaging of and/or treatment of tumors.

It is another object of the present invention to provide for methods, apparatus and products relating to the imaging of and/or treatment of somatostatin receptor or other receptor bearing tumors.

There is even another object of the present invention to provide for methods, apparatus and products relating to the radio imaging of and/or treatment of somatostatin receptor or other receptor bearing tumors.

These and other objects of the present invention will become apparent to those of skill in the art upon review of this specification, including its drawings and claims.

According to one non-limiting embodiment of the present invention, there is provided a method of treating a target cell comprising a moiety receptor. The method may include contacting the target cell with a radiolabeled moiety analog having the formula [⁸⁹Zr]-moiety. In non-limiting sub-embodiments, the target cell comprises a somatostatin receptor and the formula comprises [⁸⁹Zr]-somatostatin analog.

According to another non-limiting embodiment of the present invention, there is provided a method of imaging a target cell comprising a moiety receptor. The method may include contacting the target cell with a radiolabeled moiety analog in having the formula [⁸⁹Zr]-moiety, to form a radiolabeled target cell. The method may also include detecting radiation emitted by the radiolabeled target cell. The method may also include creating an image from the radiation emitted. In non-limiting sub-embodiments, the target cell comprises a somatostatin receptor and the formula comprises [⁸⁹Zr]-somatostatin analog.

According to even another non-limiting embodiment of the present invention, there is provided a method of forming radiolabeled somatostatin. One non-limiting method may include contacting a somatostatin with a radio-label in the form of ⁸⁹ZrX, wherein X is a halide selected from the group consisting of F, Cl, Br, I and At, to form a radiolabeled somatostatin of the form ⁸⁹Zr-somatostatin. Another non-limiting method for binding ⁸⁹Zr to any suitable pharmaceutical is the complexation of desferrioxamine (DFO) and zirconium and the subsequent linkage to a suitable pharmaceutical.

These and other embodiments of the present invention will become apparent to those of skill in the art upon review of this specification, including its drawings and claims.

### DETAILED DESCRIPTION OF THE INVENTION

The imaging methods of the present invention relate to nuclear imaging that generally involves the detection of a radio tracer that has been introduced into the subject to be detected. Certainly, while in the practice of the present invention, any suitable radio tracer may be utilized, a non-limiting example of a suitable radio tracer for the present invention will include any radio tracer comprising ⁸⁹Zr. More specifically, in the imaging of somatostatin receptor bearing targets, radio tracers suitable for the present invention may comprise a somatostatin, meaning somatostatin or any chemical analogue thereof. Even more specifically, as further non-limiting examples, radio tracers suitable for the present invention may comprise both⁸⁹Zr and a somatostatin.

The imaging and or treatment methods of the present invention involve active transport of the radioactively conjugated moiety into cancer cells. Once localized, the tumor can be imaged with a positron emission tomography (PET) scanner. As with the imaging methods of the present invention, any suitable radiation emitting material may be utilized. A non-limiting example of a suitable radiation emitting material for the present invention will include any radiation emitting material comprising ⁸⁹Zr. More specifically, in the treatment of somatostatin receptor bearing tumors, radiation emitting materials suitable for the present invention may comprise a somatostatin, meaning somatostatin or any chemical analogue thereof. Even more specifically, as further non-limiting examples, radio tracers suitable for the present invention may comprise both⁸⁹Zr and a somatostatin. Additional pharmaceutical moieties may include but are not limited to all radiopharmaceutical agents currently approved for use, allowing for substitution of the previously approved isotope for ⁸⁹Zr.These pharmaceutical categories include but are not limited to sestamibi, medronate, texboroxamine, exametamine, MIBG, mebrofenin, pentetate, gluceptate, mebrofenin, disofenin, lidofenin, MAA, sulfur colloid, pyrophosphate, citrate, oxyquinoline, pentetate disodium, mertiatide, capromabpendetide, satumomabpendetide, as well as other currently approved radiopharmaceuticals.

In the practice of the present invention, any suitable radio tracer or radiation emitting material comprising a somatostatin and ⁸⁹Zr may be bound to the somatostatin utilizing any suitable method. Generally, when utilized as a halide, ⁸⁹ZrX may be bound in the same manner as ¹¹¹InCl or ¹⁷⁷LuCl which have in the prior art been bound to this type of peptide. Suitable halides include F, Cl, Br, I and At. Commonly in the present invention it is anticipated that ⁸⁹ZrCl would be utilized. A second method for binding ⁸⁹Zr to any suitable pharmaceutical is the complexation of desferrioxamine (DFO) and zirconium and the subsequent linkage to a suitable pharmaceutical. Several coupling strategies are described in D. J. Vugts et al., “⁸⁹Zr-labelled compounds for PET imaging guided personalized therapy, in Drug Discovery Today: Technologies, Vol. 8, 2-4, pp e53-e61(2011)” where ⁸⁹Zr-DFO was linked to monoclonal antibodies with good binding affinities.

Radiolabeled analogs of the peptide somatostatin have been studied for their effectiveness in tumor imaging and therapy. See E. A. Woltering et al., “The Role of Radiolabelled Somatostatin Analogs in the Management of Cancer Patients,” Principles & Practice of Oncology, Vol. 9, pp. 1-15 (1995); U.S. Pat. No. 5,590,656; and U.S. Pat. No. 5,597,894. Endogenously produced somatostatin, a tetradecapeptide, inhibits release of several pituitary and intestinal factors that regulate cell proliferation, cell motility, or cellular secretion, including growth hormone, adrenocorticotropin hormone, prolactin, thyroid stimulating hormone, insulin, glucagon, motilin, gastric inhibitory peptide (GIP), vasoactive intestinal peptide (VIP), secretin, cholecystokinin, bombesin, gastrin releasing peptide (GRP), gastrin adrenocorticotropic hormone (ACTH), thyroid releasing hormone (TRH), cholecystokinin (CCK), aldosterone, pancreatic polypeptide (PP), various cytokines (e.g., interleukins, interferons), various growth factors (e.g., epidermal growth factor, nerve growth factor), and various vasoactive amines (e.g., serotonin).

Because somatostatin has a short biologic half-life (1 to 2 min), a variety of somatostatin peptide analogs have been produced by elimination of amino acids, by substitution of native L-amino acids with the corresponding D-amino acid isomers, by addition of an alcohol to the carboxy terminus of the molecule, or by various combinations of these approaches. See U.S. Pat. No. 5,597,894. Examples of somatostatin analogs include octreotide acetate, lanreotide, vapreotide (“RC-160”), and pentetreotide, all which have a longer biologic half-life. Multi-tyrosinatedsomatostatin analogues have been produced and shown to bind somatostatin cellular receptors. See U.S. Pat. No. 5,597,894.

Somatostatin receptors are found throughout the cell, including the cell membrane, Golgi apparatus, endoplasmic reticulum, vesicles, and nucleus. Somatostatin and its analogs are internalized by endocytosis of the ligand-receptor complex. See L. J. Hofland et al., “Internalization of the RadioiodinatedSomatostatin Analog [.sup.125 I-Tyr.sup.3]Octreotide by Mouse and Human Pituitary Tumor Cells: Increase by Unlabeled Octreotide,” Endocrinology, vol. 136, pp. 3698-3706 (1995); Wiseman et al., (1995).

High densities of somatostatin receptors, especially somatostatin receptor subtype 2 (SST-2), have been found on cells from a wide variety of tumors, including endocrine tumors, melanomas, breast carcinomas, Merkel cell tumors, lymphomas, small cell lung carcinomas, gastrointestinal tumors, astrocytomas, gliomas, meningiomas, carcinoid tumors, islet cell tumors, renal cell carcinomas, neuroblastomas, and pheochromocytomas. See E. A. Woltering et al., “The Role of Radiolabeled Somatostatin Analogs in the Management of Cancer Patients,” Principles & Practice of Oncology, Vol. 9, pp. 1-15 (1995); and E. A. Woltering et al., “Somatostatin Analogs: Angiogenesis Inhibitors with Novel Mechanisms of Action,” Investigational New Drugs, vol. 15, pp. 77-86 (1997). The radiolabeled somatostatin analog .sup.111 In-Pentetreotide, known to bind SST-2 receptors on cell membranes, has been shown to bind to pituitary tumors, endocrine pancreatic tumors, carcinoids, paragangliomas, pheochromocytomas, medullary thyroid carcinomas, small-cell-lung cancers, neuroblastomas, meningiomas, breast carcinomas, renal cell carcinomas, gliomas, astrocytomas, melanomas, and lymphomas. .sup.111 In-Pentetreotide has also been used to treat metastatic glucagonoma and carcinoid tumors. See Wiseman et al., 1995; Krenning et al., “Radiotherapy with a radiolabelled somatostatin analogue, [.sup.111 In-DTPA-D-Phel]-octreotide. A Case History,” Annals of the New York Academy of Sciences, vol. 733, pp. 496-506 (1996); and M. Fjalling et al., “Systemic radionuclide therapy using indium-111-DTPA-D-Phe-1-octreotide in midgut carcinoid syndrome,” Journal of Nuclear Medicine, vol. 37, pp. 1519-21 (1996).

Radiolabeled somatostatin or somatostatin analogs have been used for tumor imaging and therapy, but have previously been administered either by bolus injection or by short infusion (up to 2 hours). See S. W. J. Lamberts et al., “Somatostatin-Receptor Imaging in the Localization of Endocrine Tumors,” The New England Journal of Medicine, vol. 323, pp. 1246-49 (1990); E. P. Krenning et al., “Somatostatin Receptor Scintigraphy with Indium¹¹¹-DTPA-D-Phe¹-Octreotide in Man: Metabolism, Dosimetry and Comparison with Iodine123-Tyr-3-Octreotide,” The Journal of Nuclear Medicine, vol. 33, pp. 652-58 (1992); E. P. Krenning et al., “Localisation of Endocrine-Related Tumours with Radioiodinated Analogue of Somatostatin,” The Lancet, vol. 1989, no. 1, pp. 242-244 (1989); W. A. P. Breeman et al., “Studies on Radiolabelled Somatostatin Analogues in Rats and in Patients,” The Quarterly Journal of Nuclear Medicine, vol. 40, pp. 209-220 (1996); and E. P. Krenning et al., “Somatostatin Receptor Scintigraphy with [¹¹¹ In-DTPA-D-Phe¹]- and [¹²³ I-_{Tyr}3]-octreotide: the Rotterdam Experience with More than 1000 Patients,” European Journal of Nuclear Medicine, vol. 20, pp. 716-31(1993). Any of these disclosed somatostatins are believed to be useful in the radiolabeled ⁸⁹Zr-somatostatin analogs of the present invention.

Non-limiting examples of suitable somatostatins include DTPA-pentetreotide, DOTA-TYR³-octreotate, ((DTPA-D-Phe¹)-octreotide), (DOTA⁰-D-Phe¹-Tyr³)-octreotide, (DTPA-D-Phe¹)-RC-160, RC-160, CPTA-RC-160, octreotide, tyr³-octreotide, lanreotide, DOTA-lanreotide, DPTA-lanreotide, DPTA- somatostatin, DOTA- somatostatin, somatostatin, WOC-3b, WOC-4a, JIC-2D, and WOC-3.

Other chemical suitable for use in the present invention include but are not limited to the sterols, estrogen, progesterone, and dexametazone, testosterone, amines other than somatostatin, any peptide that has a cell surface receptor, VIP (see Virgolini), gastrin, glucaogon, growth hormone, and all other gastro endo-pancreatic GEP agent including gastrin receptors (see Townsend, UT Galveston),such as but not inclusive to: GEP agents, bombesin in ovarian and lung cancer. In the present invention, somatotatin to include cancer types other than carcinoid, e.g., breast cancer, ovarian cancer, thyroid cancer.

Even other chemicals suitable for use in the present invention include but are not limited to, amines to include but not limited to, dopamine, serotonin bradykinin and kalliakinin, amino acids and nucleotides including but not limited to, MIBG, guanine, adnine, uracil, cytosine and thymine, adenosine compounds (ATP, etc.), and all biochemical moities with surface or cytoplasmic or nuclear receptors.

As a non-limiting example, one method of preparing such a radiolabeled ⁸⁹Zr-somatostatin analog is by the complexation of a ⁸⁹Zr-halide with the desired somatostatin, for example, the complexation of a [⁸⁹Zr]-chloride with a somatostatin.

The following are non-limiting examples somatostatin analogs suitable for use in the present invention, [⁸⁹Zr]-DTPA-pentetreotide, [⁸⁹Zr]-DOTA-TYR³-octreotate, (([⁸⁹Zr]-DTPA-D-Phe¹)-octreotide), ([⁸⁹Zr]-DOTA⁰-D-Phe¹-Tyr³)-octreotide, ([⁸⁹Zr]-DTPA-D-Phe¹)-RC-160, [⁸⁹Zr]-RC-160, [⁸⁹Zr]-CPTA-RC-160, [⁸⁹Zr]-octreotide, [⁸⁹Zr]-tyr³-octreotide, [⁸⁹Zr]-lanreotide, [⁸⁹Zr]-DOTA-lanreotide, [⁸⁹Zr]-DPTA-lanreotide, [⁸⁹Zr]-DPTA-somatostatin, [⁸⁹Zr]-DOTA-somatostatin, [⁸⁹Zr]-somatostatin, [⁸⁹Zr]-WOC-3b, [⁸⁹Zr]-WOC-4a, [⁸⁹Zr]-JIC-2D, and [⁸⁹Zr]-WOC-3.

Positron emission tomography (PET) is a very well known nuclear medicine imaging technique that produces a three-dimensional image or picture of functional processes in the body. It is not known to utilize an ⁸⁹Zr-somatostatin in the practice of PET. In general, the PET system detects pairs of gamma rays emitted indirectly by a positron-emitting radionuclide (tracer), which is introduced into the body on a biologically active molecule. Three-dimensional images of tracer concentration within the body are then constructed by computer analysis. Very commonly, three dimensional imaging is often accomplished with the aid of a CT X-ray scan performed on the patient during the same session, in the same machine.

As a non-limiting embodiment of the present invention, to conduct the scan, a short-lived radioactive tracer isotope is injected into the living subject (usually into the blood stream). The tracer, a non-limiting example of which is ⁸⁹Zr, is chemically incorporated into a biologically active molecule, as a non-limiting example of which is a somatostatin. There is a waiting period on the order of minutes to hours while the active molecule becomes concentrated in tissues of interest; then the subject is placed in the imaging scanner. During the scan a record of tissue concentration versus time is made as the tracer decays.

As a non-limiting embodiment of the present invention, a treatment method of the present invention includes contacting a radiolabeled ⁸⁹Zr-somatostatin analogs with target cells comprising a complimentary somatostatin receptor.

### EXAMPLES

The following non-limiting examples are hereby provided to illustrate some of the embodiments of the present invention, and they are not meant to illustrate all of the embodiments of the present invention, nor are they intended to limit and they do not limit the invention, nor the scope of the claims.

Labeling and Testing of ⁸⁹Zr

10 mCi was obtained from Ion Beam Applications (IBA) pharmaceuticals in the Netherlands. Samples were subsequently labeled to DOTA-TYR³-octreotate as well as DTPA-Pentetreotide. Next, these conjugated radiopharmaceuticals were placed in a cell media containing (somatostatin receptor bearing) IMR 32 neuroblastoma cells. It is noted that these cells have an abundance of somatostatin receptors and will bind and internalize the radioligand.

To demonstrate that this activity was internalized, a standard laboratory analysis for binding effectiveness was utilized. Specifically, a second set of identical plates was exposed to the same quantity of radiopharmaceutical as well as a 10-6 molar concentration of Octreotide Acetate (OA). This “cold” analogue competes with the radiolabelled one for sites on the cells. A reduction in uptake for the second test demonstrates that competable cellular binding occurred in both cell media. Both somatostatin analogues demonstrated competeable binding demonstrated by a reduction in uptake by 25-35% in the OA matched samples. As a control, the same test was performed on these cells using 111In-Pentetreotide, with similar results observed.

Phantom Testing

To satisfy whether this isotope will image in a patient, phantom testing was carried out on a Phillips PET/CT unit, using criteria established by the American College of Radiology. The activity was quantitately imaged in the phantom at levels down to 0.02 uCi/cc and continued imaging for 10 days post dosing. These results show that this drug will allow for the better diagnosis of these patients as well as the detection and localization of smaller tumors as it remains tumor bound while any non-bound activity will be biologically eliminated.

Patient Population Background

Neuroendocrine tumors (NET) are a rare, heterogeneous group of malignancies. Typically, this category of malignancies is difficult to diagnose and treat. While surgery can sometimes provide a curative treatment for patients with localized disease, NETs often present as late stage tumors with advanced metastatic disease, making surgical resection impossible. Traditional chemotherapy and radiation treatment regimens may not be beneficial for patients with disseminated NETs. Therapeutic options for these patients are limited, with treatment often focused on palliative treatments.

A common feature of differentiated NETs is a high level expression of somatostatin receptors on their cell surface. This feature has been exploited by the use of radiolabeled somatostain analogues as a tool for diagnosis and treatment for NETs. Scintigraphy with ¹¹¹In labeled somatostatin analogue pentetreotide is a common method of detection of somatostatin receptor positive NETs. Additionally, ⁶⁸Ga labeled somatostatin analogues have also been used for PET-CT scan in these patients recently.

Drug Conjugation

10 mCi of [⁸⁹Zr]-oxalate solution in 1.0 M oxalic acid was obtained from Ion Beam Applications (IBA) . Aliquots of this activity were loaded onto an activated Waters Sep-pak Light QMA strong anion exchange cartridge (an acrylic acid/acrylamide copolymer on Diol silica. Surface functionality: —C(O)NH(CH₂)₃N(CH₃)₃+Cl—, 300 Å pore size, 37-55 μm particle size, 0.22 mmol/g ligand density), pre-washed with 6 mL acetonitrile (MeCN), 10 mL 0.9% saline and 10 mL water. In the exchange, >99.9% of the ⁸⁹Zr activity remained trapped on the cartridge. The cartridge was then washed with water (>40 mL) and eluted with 1.0 M HCl(aq.) (300-500 μL). The resulting [⁸⁹Zr]-chloride can be diluted in either water, 0.9% saline for ease of handling and a resultant 0.1 M HCl solution.

[⁸⁹Zr]-DTPA-Pentetreotide was prepared by the complexation of [⁸⁹Zr]-chloride (in 0.1 M HCl(aq.)) with DTPA-Pentetreotide. Aliquots of sterile [⁸⁹Zr]-chloride were diluted in 100 μL of 0.9% saline (NaCl, 4.5 g, 77 mmol, dissolved in 500 μL of water) for a resultant 0.1 M HCl solution . Following the Octreoscan® package insert as a guide, the acidic (pH 3.8-4.5) [⁸⁹Zr]-chloride was added to a sterile lyophilized pellet containing 10 μm of peptide. The vial was swirled and reaction mixture was vortexed for 30 s and left to react at room temperature for between 5-15 min. The excess 0.1 M HCl was neutralized by adding the appropriate volume of Na₂CO₃(aq.) (1.0 M or 0.1 M in water 0 and the pH adjusted to 7-8 with <1 μL portions of 0.1 M HCl(aq.) and 0.1 M Na₂CO₃(aq.). Samples were withdrawn at suitable time points and analyzed by Sep Pac C18 analysis of binding.

1. A method of treating a target cell comprising a moiety receptor, comprising:
Binding the target cell with a radiolabeled binder moiety having the formula [⁸⁹Zr]-binder moiety.

2. The method of claim 1, wherein the moiety receptor is a somatostatin receptor and the binder moiety is a somatostatin analog.

3. The method of claim 2, wherein the radiolabeled binder moiety is selected from the group consisting of: [⁸⁹Zr]-DTPA-pentetreotide, [⁸⁹Zr]-DOTA-TYR³-octreotate, (([⁸⁹Zr]Zr-DTPA-D-Phe¹)-octreotide), ([⁸⁹Zr]-DOTA⁰-D-Phe¹-Tyr³)-octreotide, ([⁸⁹Zr]-DTPA-D-Phe¹-RC-160, [⁸⁹Zr]-RC-160, [⁸⁹Zr]-CPTA-RC-160, [⁸⁹Zr]-octreotide, [⁸⁹Zr]-tyr³-octreotide, [⁸⁹Zr]-lanreotide, [⁸⁹Zr]-DOTA-lanreotide, [⁸⁹Zr]-DPTA-lanreotide, [⁸⁹Zr]-DPTA-somatostatin, [⁸⁹Zr]-DOTA-somatostatin, [⁸⁹Zr]-somatostatin, [⁸⁹Zr]-WOC-3b, [⁸⁹Zr]-WOC-4a, [⁸⁹Zr]-JIC-2D, and [⁸⁹Zr]-WOC-3.

4. A method of imaging a target cell comprising a moiety receptor, comprising:
Binding the target cell with a radiolabeled binder moiety having the formula [⁸⁹Zr]-moiety, to form a radiolabeled target cell;
Detecting radiation emitted by the radiolabeled target cell; and
Creating an image from the radiation emitted.

5. The method of claim 4, wherein the moiety receptor is a somatostatin receptor and the binder moiety is a somatostatin analog.

6. The method of claim 5, wherein the radiolabeled binder moiety is selected from the group consisting of: [⁸⁹Zr]Zr-DTPA-pentetreotide, [⁸⁹Zr]Zr-DOTA-TYR3-octreotate, (([⁸⁹Zr]Zr-DTPA-D-Phe¹)-octreotide), ([⁸⁹Zr]Zr-DOTA° -D-Phe'-Tyr³)-octreotide, ([⁸⁹Zr]Zr-DTPA-D-Phe¹)-RC-160, [⁸⁹Zr]Zr-RC-160, [⁸⁹Zr]Zr-CPTA-RC-160, [⁸⁹Zr]Zr-octreotide, [⁸⁹Zr]Zr-tyr³-octreotide, [⁸⁹Zr]Zr-lanreotide, [⁸⁹Zr]Zr-DOTA-lanreotide, [⁸⁹Zr]Zr-DPTA-lanreotide, [⁸⁹Zr]Zr-DPTA-somatostatin, [⁸⁹Zr]Zr-DOTA-somatostatin, [⁸⁹Zr]Zr-somatostatin, [⁸⁹Zr]Zr-WOC-3b, [⁸⁹Zr]Zr-WOC-4a, [⁸⁹Zr]Zr-JIC-2D, and [⁸⁹Zr]Zr-WOC3.

5. A method of forming radiolabeled moiety, comprising:
Binding a moiety with a radio-label comprising ⁸⁹Zr to form a radiolabeled moiety in the form of ⁸⁹Zr-moiety.

6. The method of claim 5, wherein the binding comprises complexation of desferrioxamine and ⁸⁹Zr with the subsequent linkage to the moiety.

7. The method of claim 6, wherein the moiety is a somatostatin is selected from the group consisting of DTPA-pentetreotide, DOTA-TYR3-octreotate, ((DTPA-D-Phe¹)-octreotide), (DOTA⁰-D-Phe¹-Tyr³)-octreotide, (DTPA-D-Phe¹)-RC-160, RC-160, CPTA-RC-160, octreotide, tyr³-octreotide, lanreotide, DOTA-lanreotide, DPTA-lanreotide, DPTA-somatostatin, DOTA-somatostatin, somatostatin, WOC-3b, WOC-4a, JIC-2D, and WOC-3.

8. The method of claim 5, wherein the radio-label comprises the form of ⁸⁹ZrX, wherein X is a halide selected from the group consisting of F, Cl, Br, I and At, and the moiety is a somatostatin, to form a radiolabeled somatostatin of the form ⁸⁹Zr-somatostatin.

9. The method of claim 8, wherein the somatostatin is selected from the group consisting of DTPA-pentetreotide, DOTA-TYR3-octreotate, ((DTPA-D-Phe¹)-octreotide), (DOTA⁰-D-Phe¹-Tyr³)-octreotide, (DTPA-D-Phe¹)-RC-160, RC-160, CPTA-RC-160, octreotide, tyr³-octreotide, lanreotide, DOTA-lanreotide, DPTA-lanreotide, DPTA-somatostatin, DOTA-somatostatin, somatostatin, WOC-3b, WOC-4a, JIC-2D, and WOC-3.

10. The method of claim 9, wherein X is Cl, and the somatostatin is selected from the group consisting of DTPA-pentetreotide, DOTA-TYR3-octreotate, ((DTPA-D-Phe¹)-octreotide), (DOTA⁰-D-Phe'-Tyr³)-octreotide, (DTPA-D-Phe¹)-RC-160, RC-160, CPTA-RC-160, octreotide, tyr³-octreotide, lanreotide, DOTA-lanreotide, DPTA-lanreotide, DPTA-somatostatin, DOTA-somatostatin, somatostatin, WOC-3b, WOC-4a, JIC-2D, and WOC-3.

11. The method of claim 9, wherein the radio-label is [⁸⁹Zr]Cl, and the somatostatin is selected from the group consisting of DTPA-pentetreotide, DOTA-TYR3-octreotate, ((DTPA-D-Phe¹)-octreotide), (DOTA⁰-D-Phe¹-Tyr³)-octreotide, (DTPA-D-Phe¹)-RC-160, RC-160, CPTA-RC-160, octreotide, tyr³-octreotide, lanreotide, DOTA-lanreotide, DPTA-lanreotide, DPTA-somatostatin, DOTA-somatostatin, somatostatin, WOC-3b, WOC-4a, JIC-2D, and WOC-3, and radiolabeled somatostatin of the form [⁸⁹Zr]-somatostatin.
